(19) United States
(12) Patent Application Publication
(10) Pub. No.: US 2006/0165761 A1
(43) Pub. Date: Jul. 27, 2006

(54) Wound dressings comprising vanadate complexed with organic ligands

(76) Inventor: Patrick Trotter, Meanwood (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/538,315

(22) PCT Filed: Dec. 8, 2003
(86) PCT No.: PCT/GB03/05336

§ 371 Date: Feb. 27, 2006

(30) Foreign Application Priority Data
Dec. 12, 2002 (GB) 0229012.0

(51) Int. Cl. A61L 15/00; A61K 31/555

(52) U.S. Cl. 424/445; 514/184

(57) ABSTRACT

The invention relates to wound dressings comprising a tyrosine phosphatase inhibitor that stimulates collagen deposition and fibroblast proliferation thereby accelerating wound healing. Preferred tyrosine phosphatase inhibitors are complexes of vanadate and peroxovanadate with organic ligands.

The present invention relates to wound dressings and to compositions for use in wound healing.

Growth factors such as epidermal growth factor (EGF), platelet derived growth factor (PDGF) and fibroblast growth factor (FGF) play an important role in wound healing. These molecules act by binding to receptors on the cell surface. This binding induces conformational changes in the growth factor receptors, which in turn activates their intrinsic kinase activity and causes the phosphorylation of many intracellular proteins, including phospholipase C-g and PI-3 kinase.

In cells involved in wound healing (fibroblasts, keratinocytes, endothelial cells etc.) it is this signal transduction pathway that causes an increase in cell proliferation, the deposition of components of the extracellular matrix and is the molecular basis of the wound healing process.

Protein tyrosine phosphatases (PTP) are proteins that reverse the effects of protein kinases by a process called dephosphorylation. These proteins act as molecular switches that can switch off activation signals. Indeed the non-specific, cell permeable protein tyrosine phosphatase inhibitor vanadate has been shown to cause bone cell proliferation and bone collagen synthesis in in vitro studies (Lau, K. H. Tanimoto, H. and Baylink, D. K. (1988) Vanadate stimulates cell proliferation and bone collagen synthesis in vitro. Endocrinology. 123, 2858-67). In addition in capillary endothelial cells vanadate has been shown to mimic the effects of fibroblast growth factor in that it stimulates endothelial cells to invade collagen matrices and organise into tubules (Montesano, R., Pepper, M. S., Belin, D., Vassalli, J. D., and Orci, L. describe induction of angiogenesis in vitro by vanadate, an inhibitor of phosphotyrosine phosphatases (1988) J. Cell. Physiol. 134: 460-6).

U.S. Pat. No. 5,741,777 (to Grinnell et al.) describes the modulation of wound contraction by blocking protein tyrosine phosphatase using tyrosine phosphatase inhibitors in the presence of a growth factor. No mention of increased cell proliferation is made and moreover U.S. Pat. No. 5,741,777 employs a non-specific protein phosphatase (pervanadate). U.S. Pat. No. 5,741,777 also states that because of the involvement of tyrosine kinases and tyrosine phosphatases in the regulation of cell growth, inhibition of tyrosine phosphatases is likely to be a promising means of inhibiting cell growth in general.

US 20020019412 A1 (to Henrik Sune Anderson et al.) discloses various uses of inhibitors of PTPases, e.g. in treating diabetes. However, the use of PTPase inhibitors in wound dressings is not disclosed.

Ehrich, H. P., Keefer, K. A., Maish, G. O. 3rd, Myers, R. L. and Mackay, D. R. in Plast Reconstr. Surg. 107(2), 471-7 (2001) disclose that vanadate ingestion increases the gain in wound breaking strength and leads to better organised collagen fibres during healing of acute wounds in rats. Vanadate inhibits the activity of a number of enzymes, including tyrosine phosphatase. There is no suggestion topical application of the vanadate, for example as or in a wound dressing.

Vanadate and pervanadate are non-specific tyrosine phosphatase inhibitors. That is they inhibit all tyrosine phosphatase enzymes and are not selective. They therefore modify many cellular processes and offer no control over what signal transduction pathways are modified. For example, Vanadate (Sodium Orthovanadate) is a broad spectrum inhibitor of protein tyrosine phosphatases. It is also known to inhibit Na⁺/K⁺ ATPase, acid and alkalinephosphatases, phosphofructokinase, and adenylate cyclase. It is therefore not selective in its molecular target and as such the specific cellular response following addition of vanadate is complex and is difficult to predict.

Pervanadate is a derivative of vanadate and can be made by adding H₂O₂ to vanadate. Pervanadate is more potent than vanadate due to having a greater ability to migrate through the cell membrane into the cell. It is however also very non-specific and as such is toxic. The toxicity can be explained by its low specificity, it inhibits all tyrosine phosphatases as well as other essential enzymes such as Na⁺/K⁺ ATPase, acid and alkaline phosphatases, phosphofructokinase, and adenylate cyclase.

It is desired to devise tyrosine phosphatase inhibitors having higher specificity than orthovanadate or pervanadate that are likely to affect only the signal transduction pathways that would be activated by natural growth factors. Unwanted side effects are thus minimised.

We have now discovered that certain organic complexes of vanadate greatly increase the rate of cell proliferation, collagen deposition and ultimately the rate of wound healing if used in a clinical situation. The effect is significantly greater that that observed for vanadate alone. Furthermore, the organic nature of these complexes means that they can be tailored to provide the desired absence of toxicity and side effects.

Accordingly, a first aspect of the invention provides a wound dressing comprising a complex of vanadate with an organic ligand, wherein said complex stimulates collagen deposition and fibroblast proliferation.

The vanadate is normally a vanadate (V) complex. It may be mononuclear or it may be a polynuclear complex. It may also be a pervanadate complex, i.e a complex of vanadate (V) with peroxide and the organic ligand. It will be appreciated that the vanadate complex will normally carry an overall ionic charge, normally a cationic charge, and that it will then be associated with a suitable pharmaceutically acceptable counterion such as sulfate, chloride, (hydrogen) phosphate, p-toluene sulfonate, acetate, trifluoroacetate, propionate, citrate, malonate, succinate, lactate, oxalate, tartarate, benzoate, or mixtures thereof. The uses and products of the present invention encompass all medically acceptable salts, isomers and analogs of the claimed vanadate complexes.

The ligand is an organic ligand. That is to say, it comprises at least one carbon atom and at least one C—H bond. However, preferably the complex is not an organometallic complex, i.e. a complex having direct bonding between vanadium and carbon. The ligand is preferably bonded to the vanadium through a nitrogen atom, for example an amine nitrogen (primary, secondary or tertiary), imine nitrogen, or aromatic nitrogen such as pyridine or pyrrole. In other preferred embodiments, the ligand is bonded to the vanadium through an anionic oxygen atom, for example a carboxylate oxygen or amido oxygen. In certain preferred embodiments, both such ligands are present in the complex.

Vanadate tends to form more stable complexes with strong σ-donor ligands than with weak σ-donors, and more stable complexes with π-donor ligands than with π-acceptor ligands. It has been found that more stable complexes appear to exhibit higher activity in the assay for collagen deposition and fibroblast proliferation. Accordingly, the complexes used in the present invention preferably comprise strong σ-donor ligands, such as nitrogen ligands and anionic oxygen ligands. Ligands exhibiting π-donor characteristics, such as anionic oxygen, pyrrole and imidazole are preferred over π-acceptor ligands such as nitrile, isonitrile, and most pyridine-type heterocycles, including bipyridyl and phenanthroline, (but not including pyridine-type heterocycles having π-donor substituents). For the same reason, the complex of vanadate with an organic ligand is preferably a complex of vanadate with one or more multidentate organic ligands, more preferably a bidentate organic ligand.

Since tyrosine contains a single aromatic ring, it follows that tyrosine phosphatases have receptor sites that are especially likely to bind inhibitors molecules having a single aromatic ring, and accordingly the complex of vanadate with an organic ligand is preferably a complex of vanadate with one or more organic ligands containing a single (optionally substituted as defined below) aromatic ring, more preferably a phenyl ring.

Preferably, the organic ligand is selected from the group consisting of:

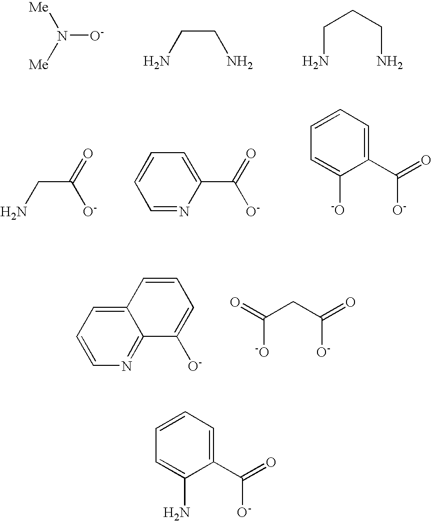

Preferably, one or more of the Carbon atoms on the organic ligand is optionally substituted with substituents selected from: hydrogen; halogen atoms, e.g. fluorine, chlorine, bromine or iodine atoms; C₁₋₆ alkyl, e.g. methyl or ethyl; C₁₋₆ alkoxy e.g. methoxy or ethoxy; C₂₋₆ alkylenedioxy, e.g. ethylenedioxy; haloC₁₋₆alkyl, e.g. tri-fluoromethyl; C₁₋₆ alkylamino, e.g. methylamino or ethylamino; C₁₋₆ dialkylamino, e.g. dimethylamino or diethylamino; amino (—NH₂); nitro; cyano; hydroxyl (—OH); carboxyl (—CO₂H); ester —CO₂R, where R is preferably C₁₋₆ alkyl; C₁₋₆alkylcarbonyl, e.g. acetyl; sulfonyl (—SO₂H); C₁₋₆alkylsulfonyl, e.g. methylsulphonyl; aminosulphonyl (—SO₂NH₂); C₁₋₆alkylaminosulphonyl e.g. methylaminosulphonyl or ethylaminosulphonyl; C₁₋₆dialkylaminosulphonyl e.g. dimethypaminosulphonyl or diethylaminosulphonyl; carboxamido (—CONH2); C₁₋₆alkylaminocarbonyl, e.g. methylaminocarbonyl or ethylaminocarbonyl; C₁₋₆ dialkylaminocarbonyl, e.g. dimethylaminocarbonyl or diethylaminocarbonyl; sulphonylamino (—NHSO₂H); C₁₋₆ alkylsulphonylamino, e.g. methylsulphonylamino or ethylsulphonylamino; or C₁₋₆dialkylsulphonylamino, e.g. dimethylsulphonylamino or diethylsulphonylamino groups. It will be appreciated that where two or more such substituents are present, these need not necessarily be the same atoms and/or groups. It will also be appreciated that each of the alkyl carbon atoms on any of the above substituents may itself be substituted in turn by any of the substituents defined above.

Particularly preferred ligands include picolinate or N,N-dimethylamido (i.e. (CH₃)₂N—O⁻).

Preferably, the or each vanadate complex employed in the present invention is:
(i) A member of the peroxovanadium family, preferably bpv(pic) Bisperoxo (5-hydroxy pyridine-2-carboxyvanadate) and mpV (pic) Monoperoxo(picolinato) oxovanadate, or
(ii) DHMV (Bis(N,N dimethylhydroxamido) hydroxoxovanadate.

Preferably, the vanadate complex employed is bpv (pic), bpv (bipy), or DMHV. Analogs of all these molecules could be designed to increase stability, specificity or rate of healing. Accordingly, in one preferred embodiment of the invention an analog of bpv (pic), bpv (bipy), dephostatin or DMHV is employed.

The vanadate complexes mentioned above are hydrolysed in aqueous solutions after a few days. This should prevent long term toxicological effects.

As indicated in the Examples section below, not all vanadate complexes enhance collagen deposition and fibroblast definition. For example, we have found that bpV(Phen) (i.e. bisperoxo-(1,10-phenanthroline)-oxovanadate) does not enhance collagen deposition and fibroblast proliferation. Indeed, bpV (Phen) appeared to be an inhibitor of proliferation.

In view of the fact that not all vanadate complexes enhance collagen deposition and fibroblast proliferation, and thus not all increase the rate of wound healing, it will be necessary to verify that the vanadate complex does indeed enhance collagen deposition and fibroblast proliferation. Those skilled in the art will be able to readily devise assays for determining whether a given vanadate complex enhances collagen deposition and fibroblast proliferation. For instance, it is possible to determine whether a vanadate complex enhances collagen deposition and fibroblast proliferation definition using the assay methods described in the Examples section below.

Preferably, the vanadate or pervanadate complexes used in the methods and wound dressings of the invention exhibit greater enhancement of collagen deposition and/or fibroblast proliferation definition, as determined using the assay methods described in the Examples section below, than vanadate or pervanadate alone, respectively.

It is an advantage of the present invention that the tyrosine phospatase inhibitors are generally not substrates for the proteases present in wound fluid. Accordingly, the action of the or each tyrosine phospatase inhibitor should not be affected by high protease levels which could make other similar (growth factor) treatments ineffective, especially in chronic wounds.

In certain embodiments of the invention, two, three, four or more vanadate complexes are employed. Where at least two vanadate complexes are employed, the two or more vanadate complexes may act synergistically together.

In a second aspect, the present invention provides the use of a complex of vanadate with an organic ligand, wherein the complex stimulates collagen deposition and fibroblast proliferation, in the manufacture of a medicament for increasing the rate of wound healing.

Preferably, the medicament is a wound dressing according to the first aspect of the present invention.

The term “wound” refers broadly to injuries to the skin and subcutaneous tissue initiated in different ways (e.g., pressure sores from extended bed rest and wounds induced by trauma) and with varying characteristics. Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum). The term “partial thickness wound” refers to wounds that encompass Grades I-III; examples of partial thickness wounds include burn wounds, pressure sores, venous stasis ulcers, and diabetic ulcers. The term “deep wound” is meant to include both Grade III and Grade IV wounds. The present invention contemplates treating all wound types, including deep wounds and chronic wounds. The term “chronic wound” refers to a wound that has not healed within 30 days. The delay in healing may, for example, be caused by elevated levels of matrix metalloproteinases (MMP's). Typically, the wound is a chronic wound. Preferably, it is selected from the group consisting of venous ulcers, pressure sores and decubitis ulcers.

As discussed in the experimental section below, we have found that improved results are achieved in the absence of supplementary growth factors. Thus, it is preferred that supplementary growth factors are not employed in the wound dressings, medicaments and uses of the invention.

Hence, with regard to the method of treatment according to the invention, it is preferred that the patient is not administered a growth factor under circumstances which could adversely affect the therapeutic effects of the vanadate complex(s) on wound healing. By “administered a growth factor under circumstances which could adversely affect the therapeutic effects of the vanadate complex(s)”, we include, for example, the use of a medicament or dressing comprising both a vanadate complex(s) and a growth factor. Similarly, we include the administration of a growth factor to the wound shortly before or after administration of the vanadate complex(s) where the time difference between administration of the growth factor and the vanadate complex(s) is such that the growth factor could adversely affect the therapeutic effect of the vanadate complex(s) on wound healing.

Similarly, it is preferred that the medicaments and wound dressings of the invention do not comprise a growth factor. It is also preferred that a growth factor is not administered to the wound shortly before or after administration of the medicament/wound dressing where the time difference between administration of the growth factor and the medicament/wound dressing is such that the growth factor could adversely affect the therapeutic effect of the vanadate complex(s) in the medicament/wound dressing on wound healing.

The vanadate complexes employed in the present invention modulate the first part of the signal transduction pathway. Treatment with vanadate complexes is likely to be more effective than other pharmacological/biochemical treatments that would be expected to affect downstream processes.

The term “protein tyrosine phosphatase inhibitor” refers to compounds which would cause an increase in the phosphotyrosine content of cells by a mechanism involving inhibition of tyrosine phosphatase activity. There are a variety of assays which may be used to determine if a compound is a protein tyrosine phosphatase inhibitor involving radioactive labeled cells (with phosphorous 32 or phosphorous −33 or antibodies specific to phosphotyrosine).

In a third aspect, the present invention provides a method of increasing the rate of wound healing, the method comprising administering to the patient a complex of vanadate with an organic ligand which stimulates collagen deposition and fibroblast proliferation.

It is not intended that the present invention be limited to the mode by which the vanadate complex is administered to the patient. In one embodiment, the present invention contemplates systemic administration of the compound (e.g. parenteral or oral administration). In another embodiment, the present invention contemplates topical administration, including but not limited to topical administration using solid supports (such as dressings and other matrices) and medicinal formulations (such as mixtures, suspensions and ointments). In one embodiment, the solid support comprises a biocompatible membrane. In another embodiment, the solid support comprises a wound dressing.

Similarly, it is not intended that the present invention be limited by the particular nature of the therapeutic preparation. For example, the vanadate complex can be provided together with physiologically tolerable liquid, gel or solid carriers, diluents, adjuvants and excipients.

These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts.

Such compositions are typically prepared as liquid solutions or suspensions, or in solid forms. Formulations for wound healing usually will include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained release formulations, or powders, and typically contain 1%-95% of active ingredient, preferably 2%-70%ⁱ.

The compositions are also prepared as injectables, either as liquid solutions or suspensions; solid forms suitable for solution in, or suspension in, liquid prior to injection may also be prepared.

The vanadate complex may be mixed with diluents or excipients which are physiological tolerable and compatible. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH buffering agents.

Additional formulations which are suitable for other modes of administration, such as topical administration, include salves, tinctures, creams, lotions, and, in some cases, suppositories. For salves and creams, traditional binders, carriers and excipients may include, for example, polyalkylene glycols or triglycerides.

In a fourth aspect, the present invention provides a wound dressing comprising a tyrosine phosphatase inhibitor. The term “wound dressing” in this specification refers to a dressing for topical application to a wound and excludes compositions suitable for systemic administration. For example, the tyrosine phosphatase inhibitor may be dispersed in or on a solid sheet of wound contacting material such as a woven or nonwoven textile material, or it may be dispersed in a layer of foam such as polyurethane foam, or in a hydrogel such as a polyurethane hydrogel, a polyacrylate hydrogel, gelatin, carboxymethyl cellulose, pectin, alginate, and/or hyaluronic acid hydrogel, for example in a gel or ointment. In preferred embodiments the tyrosine phosphatase inhibitor is dispersed in or on a biodegradable sheet material that provides sustained release of the tyrosine phosphatase into the wound, for example a sheet of freeze-dried collagen, freeze-dried collagen/alginate mixtures (available under the Registered Trade Mark FIBRACOL from Johnson & Johnson Medical Limited) or freeze-dried collagen/oxidized regenerated cellulose (available under the Registered Trade Mark PROMOGRAN from Johnson & Johnson Medical Limited).

In a fifth aspect, the present invention provides the use of a tyrosine phosphatase inhibitor other than orthovanadate for the preparation of a medicament for promoting the healing of wounds. Preferably, the medicament is a wound dressing. Preferably, the format of the medicament is as described above in connection with the first through fourth aspects of the invention. Preferably, the wound is a chronic wound such as a venous ulcer, a pressure sore or a diabetic ulcer.

Tyrosine phosphatase inhibitors suitable for use in the fourth and fifth aspects of the invention include the vanadate complexes of the first through third aspects of the invention. In addition, the following tyrosine phosphatase inhibitors at least could be suitable:

(1) The molecules as described and claimed by Henrik Sune et al. in US-A-20020019412, WO02/38559, WO02/04459, WO01/19830, WO01/19831, WO01/17516, WO99/46267, WO99/46268, WO99/46244, WO99/46237, WO99/46236 and WO99/15529;

(2) The Tyrosine derivatives as described and claimed by Burgess et al. in WO02/04412;

(3) The Peptides as described and claimed in WO 96/23813;

(4) Aromatic phosphonates as described and claimed by Le Blanc et al. in WO01/46204;

(5) Amino (oxo) acetic acid tyrosine phosphatase inhibitors as described and claimed by Liu et al. in WO02/18323;

(6) Napthopyrone compounds as described and claimed in WO 96/40109;

(7) O-malonyltyrosyl/o-malonyltyrosyl as described and claimed in WO 96/30332;

(8) Antibodies;

(9) Phenylsarine oxide;

(10) Periodinates as described by Leung, K. W. K., Posner, B. I. and Just, G Bioorganic and Medicinal Chemistry Letters 9, 353-356 (1998),

The disclosure of each of the documents referenced in (1)-(10) is hereby incorporated into the present specification in its entirety.

Preferably, the tyrosine phosphatase inhibitors used in the fourth and fifth aspects of the invention induce enhanced fibroblast proliferation and/or collagen deposition rates as measured according to the procedures specified below, relative to a negative control, and more preferably when measured relative to orthovanadate as positive control. Preferably, the tyrosine phosphatase inhibitors used in the fourth and fifth aspects of the invention exhibit higher specificity for tyrosine phosphatase inhibition than orthovanadate.

It will be appreciated that any alternative or preferred feature or combination of features that is disclosed herein in relation to any one aspect of the invention is hereby disclosed in relation to any other aspect of the invention.

### BRIEF DESCRIPTION OF THE FIGURES

FIG. 1 shows measured collagen deposition in the presence of a range of vanadate complexes with organic ligands, at different concentrations;

FIG. 2 shows measured fibroblast cell proliferation for the vanadate complexes and concentrations of FIG. 1; and

FIG. 3 shows a comparison of measured fibroblast cell proliferation for vanadate at 0.1 mmol and 0.01 mmol, with the same measurement made for bpv(pic) at 0.0084 mmol.

### EXAMPLES

To investigate the effect of a range of tyrosine phosphatase inhibitors on cell proliferation and collagen deposition the following experiments were performed.

Cell Treatments

Adult dermal fibroblasts were grown until 95% confluent, trypsinized (2 ml trypsin plate) to prepare a single suspension of fibroblasts. These cells were diluted in DMEM (+10% FBS) media and diluted to a concentration of 25000 cells/ml. 100 ml was placed in each well of a 96 well microplate (2500 cells/well). The fibroblasts were allowed to adhere and spread by incubating for 24 hours (at 37° C., 5% CO₂ in a humidified atmosphere) at which point the media was removed and replaced with serum free DMEM+/−stimulant/sample.

The following vanadate complexes were added in quadruplicate: bpV(pic) was added to a final concentration of 8.14 and 0.81 μM, bpV(bipy) (bisperoxo(bipyridine)oxovanadate) was added to a final concentration of 178 and 17.8 μM. bpV(phen) was added to a final concentration of 12 or 0.124 μM, DMHV (bis-dimethylhydroxamido vanadate, (Me₂N—O)₂VO(OH)) was added to a final concentration of 6.4 or 64 Mm. The vanadate complexes were all obtained as the solid potassium salts from Calbiochem, Catalog numbers 203705, 322130, and 203695.

Negative control samples were untreated and positive control samples were cells treated with DMEM containing 10% FBS or 0.15 ng/ml epidermal growth factor. As a further positive control, Dephostatin (a known tyrosine protein phosphatase inhibitor obtained from Calbiochem, catalog number 263200) was added to a final concentration of 23.8 μM.

Additional experiments were performed using the phosphatase inhibitors indicated above in the presence of epidermal growth factor (0.015 ng/ml) or platelet derived growth factor (6 ng/ml).

XTT Cell Proliferation Assay

After 72 hrs the fluid was aspirated off and 501 μl of the XTT labelling mixture (prepared by adding 0.1 ml Electron coupling reagent to every 5 mls of XTT labelling reagent) was added to each well. The plates were read at 450 nm, using a MR5000 plate reader at 2, 2.5 and 3.5 hrs. The results shown are the absorbances compared to the negative and positive controls at 3.5 hrs.

Determination of Collagen Levels

The amount of collagen produce and deposited into the extracellular environment under each experimental condition was measured using an ELISA method. Collagen type I standards (Sigma cat no 7774, lot 32K3775) and material aspirated from the fibroblasts were coated onto 96 well plates (FALCON 3072) by incubation at 37° C. for 30 min. Non-specific sites were blocked by the addition of 200 ml of 5% solution of bovine serum albumin (BSA) for 30 min. Each well was incubated with a monoclonal antibody against collagen used at a dilution of 1/2000 ((sigma cat no. 2456, lot 081K4897) overnight at 4° C. Samples were washed five times for 5 minutes in wash buffer (PBS+0.05% (v/v) TWEEN-20) and incubated with an HRP-conjugated second antibody against mouse IgG at a dilution of 1/10000 for 2 hr. Each well was washed five times in wash buffer. Deposited collagen was detected using the Sigma FAST method used according to the manufacturers instructions. The plates were incubated at room temperature for 30 minutes and read using a MR5000 plate reader at 450 nm. The standard curve was linear at 0.05-2 μg, and results are presented as μg/ml in the extracellular environment.

Results

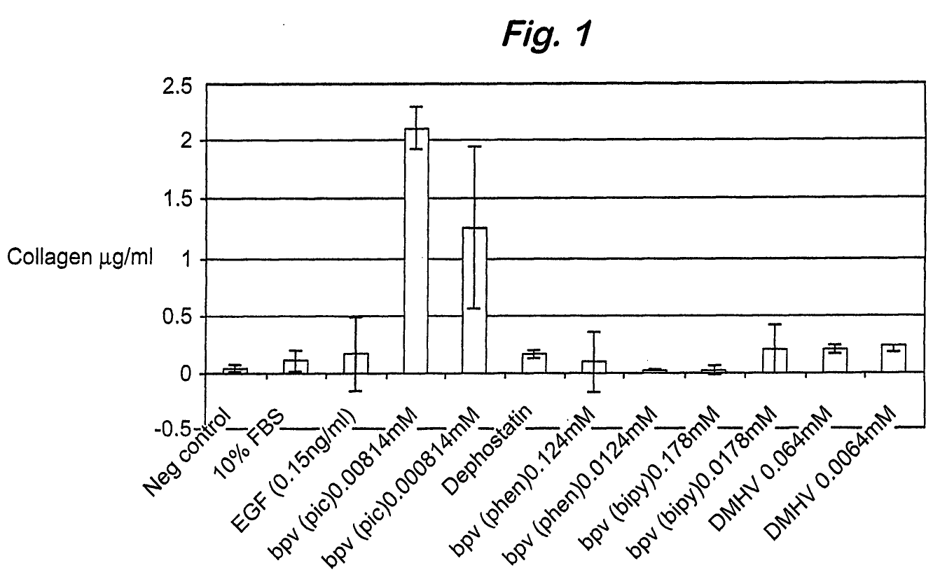

The graph in FIG. 1 shows the amount of collagen deposited by fibroblasts in the presence of phosphatase inhibitors compared to non treated (negative control, far left), and foetal bovine serum (FBS) and EGF positive controls (2^{nd} and 3^{rd} left). BpV (pic) was the most effective activator of collagen deposition at 8.14 μm. Dephostatin, (23.8 μM), bpV (bipy) (17.8 μM) and DMHV (6.4-64 μM) demonstrated rates of collagen proliferation equivalent to the positive control.

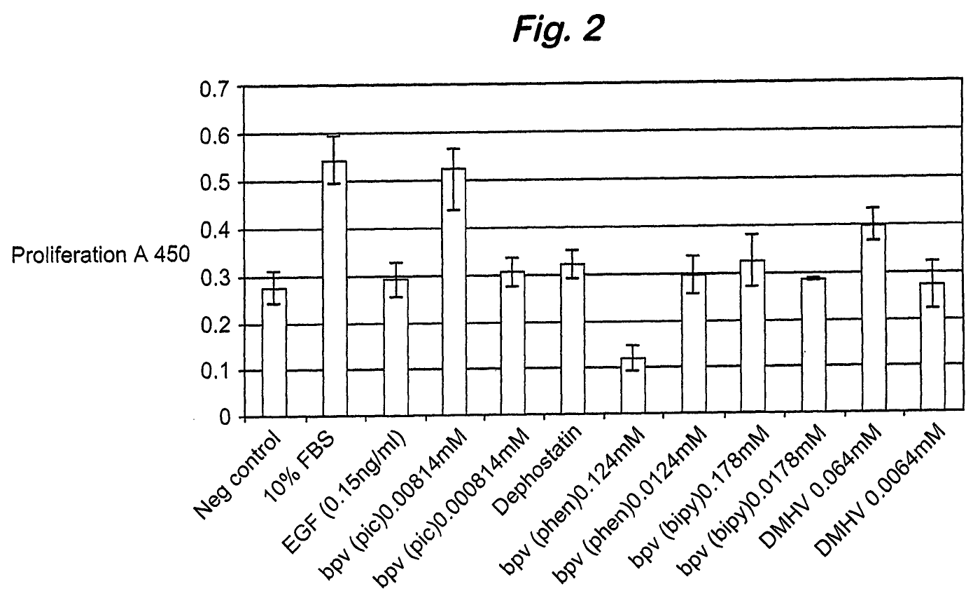

The graph in FIG. 2 shows the amount of fibroblast proliferation in the presence of phosphatase inhibitors compared to non treated (negative control, far left), and a foetal bovine serum and EGF positive controls. BpV (pic) was the most effective activator cell proliferation at 8.14 μM. This was similar to the 10% FBS positive control. Dephostatin, (23.8 μM), bpV (bipy) (17.8 μM) and DMHV (64 μM demonstrated rates of proliferation greater than the negative control.

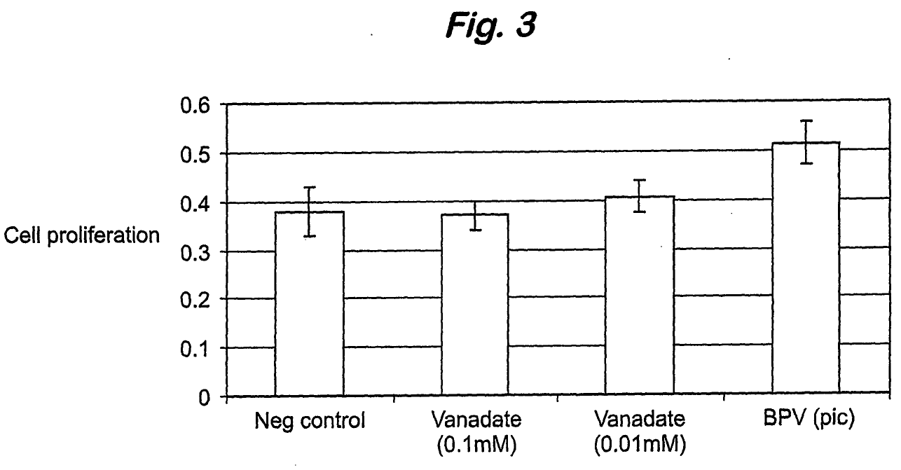

The graph in FIG. 3 shows the amount of fibroblast proliferation in the presence of bpv(pic) compared to non treated (negative control, far left), and vanadate at 0.1 and 0.01 mmolar (positive controls). BpV(pic) was the more effective activator of cell proliferation at 8.14 μM.

From these studies all the compounds tested (with the exception of bpV (phen) showed some ability to stimulate cell proliferation and collagen deposition. Bpv (pic) appeared to be more effective than even the 10% FBS positive control, while DMHV appeared to be equivalent to the DMHV positive control.

The data above show stimulation of cell proliferation and collagen deposition in the absence of growth factors. Experiments were also performed in the presence and absence of epidermal growth factor (EGF (0.015 ng/ml)) or platelet derived growth factor (6 ng/ml). The results demonstrated that cell activation did occur did still occur in the presence of phosphatase inhibitors and growth factor, but that the amount of proliferation and collagen deposition was not significantly higher than with inhibitor alone.

1. A wound dressing comprising a complex of vanadate with an organic ligand, wherein said complex stimulates collagen deposition and fibroblast proliferation.

2. A wound dressing according to claim 1, wherein the complex of vanadate with an organic ligand is a complex of vanadate with a nitrogen ligand, an oxo-anionic ligand, or a combination thereof.

3. A wound dressing according to claim 1, wherein the complex of vanadate with an organic ligand is a complex of vanadate with a π-donor ligand.

4. A wound dressing according to claim 1 wherein the complex of vanadate with an organic ligand is a complex of vanadate with a bidentate ligand.

5. A wound dressing according to claim 1, wherein the organic ligand is selected from the group consisting of:

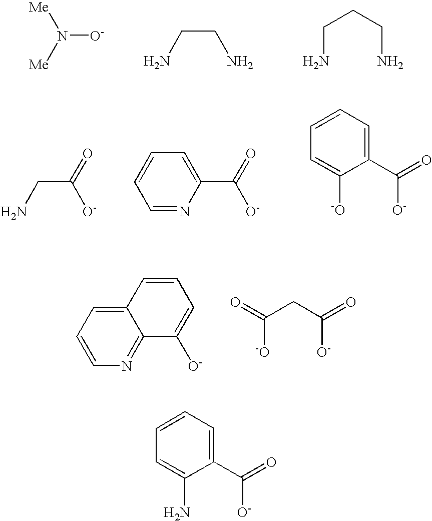

6. A wound dressing according to claim 5, wherein the organic ligand comprises picolinate or dimethylamido.

7. The wound dressing according to claim 1, wherein the wound dressing does not comprise a growth factor.

8. A wound dressing for topical application to a wound comprising a tyrosine phosphatase inhibitor.

9. A wound dressing according to claim 8, wherein the tyrosine phosphatase inhibitor is dispersed in or on a solid sheet of wound contacting material, or in a layer of foam such as polyurethane foam, or in a gel or ointment.

10. A wound dressing according to claim 9, wherein the tyrosine phosphatase inhibitor is dispersed in or on a solid sheet of biodegradable sheet material that provides sustained release of the tyrosine phosphatase into the wound.

11. A method for manufacturing a medicament for increasing the rate of wound healing comprising applying to a wound a tyrosine phosphatase inhibitor other than orthovanadate, wherein the complex stimulates collagen deposition and fibroblast proliferation.

12. The method according to claim 11, wherein the medicament is a wound dressing.

13. The method according to claim 11, wherein the wound is a chronic wound.

14. The method according to claim 13 wherein the chronic wound is selected from the group consisting of venous ulcer, pressure sore and decubitis ulcer.
